(19) United States
(12) Patent Application Publication
(10) Pub. No.: US 2024/0044863 A1
(43) Pub. Date: Feb. 8, 2024

(54) MULTI-FOCAL-PLANE SCANNING USING TIME DELAY INTEGRATION IMAGING

(71) Applicant: Applied Materials, Inc., Santa Clara, CA (US)

(72) Inventor: Christopher Bencher, Cupertino, CA (US)

(73) Assignee: Applied Materials, Inc., Santa Clara, CA (US)

(21) Appl. No.: 18/365,867

(22) Filed: Aug. 4, 2023

Related U.S. Application Data
(60) Provisional application No. 63/395,258, filed on Aug. 4, 2022.

(51) Int. Cl. G01N 33/483; G01N 21/17

(52) U.S. Cl. CPC G01N 33/4833; G01N 21/17; G01N 2021/1787; G01N 2201/1087

(57) ABSTRACT

An imaging system for capturing spatial images of biological tissue samples may include an imaging chamber configured to hold a biological tissue sample placed in the imaging system; a light source configured to illuminate the biological tissue sample to activate one or more fluorophores in the biological tissue sample; a Time Delay and Integration (TDI) imager comprising a plurality of partitions, where the plurality of partitions may be configured to capture images at a plurality of different depths in the biological tissue sample simultaneously during a scan by the TDI imager; and a controller configured to cause the TDI imager to scan the biological tissue sample.

### CROSS-REFERENCE TO RELATED APPLICATIONS

This application claims priority to Provisional U.S. Patent Application No. 63/395,258 filed Aug. 4, 2022, entitled “MULTI-FOCAL-PLANE SCANNING USING TIME DELAY INTEGRATION IMAGING,” the entire disclosure of which is hereby incorporated by reference, for all purposes, as if fully set forth herein.

### TECHNICAL FIELD

This disclosure generally describes capturing multiplexed, spatial images of biological tissue samples. More specifically, this disclosure describes camera configurations that capture multiple focal planes during a scan.

### BACKGROUND

Spatial biology is the study of the cellular and sub-cellular environment across multiple dimensions. Spatial biology tools may be used to determine which cells are present in a tissue sample, where they are located in the tissue sample, their biomarker co-expression patterns, and how these cells organize interact within the tissue sample. A sample slide may be prepared with a tissue sample in various imaging workflows may be executed to generate a comprehensive image of the tissue at the cellular and sub-cellular level, producing a single-cell resolution to visualize and quantify biomarker expression. The resulting images may expose how cells interact and organize within the tissue sample.

Capturing these complex images of the cell environment may be referred to as spatial omics. High-resolution, highly multiplexed spatial omics is rapidly becoming an essential tool in understanding diseases and other biological conditions. Typically, this type of analysis involves hundreds of complex factors, variables, and processes. An integrated solution may combine imaging and process control methods into a single machine for performing spatial omics. However, generating full spatial images of a tissue sample that accurately represent the volume of the sample requires many individual imaging scans of the sample. This large number of scans required for a full imaging analysis severely limits the throughput of the system. Therefore, improvements in the art are needed.

### SUMMARY

In some embodiments, an imaging system for capturing spatial images of biological tissue samples may include an imaging chamber configured to hold a biological tissue sample placed in the imaging system; a light source configured to illuminate the biological tissue sample to activate one or more fluorophores in the biological tissue sample; a Time Delay and Integration (TDI) imager comprising a plurality of partitions, where the plurality of partitions may be configured to capture images at a plurality of different depths in the biological tissue sample simultaneously during a scan by the TDI imager; and a controller configured to cause the TDI imager to scan the biological tissue sample.

In some embodiments, a method of capturing spatial images of a biological tissue sample may include mounting a biological tissue sample in an imaging chamber of an imaging system; directing light from a light source to illuminate an area on the biological tissue sample to activate one or more fluorophores in the biological tissue sample; and scanning the biological tissue sample with a Time Delay and Integration (TDI) imager comprising a plurality of partitions, where the plurality of partitions may be configured to capture images at a plurality of different depths in the biological tissue sample simultaneously during the scan.

In some embodiments, an imaging system may include a Time Delay and Integration (TDI) imager comprising a plurality of partitions, where the plurality of partitions may be configured to capture images at a plurality of different depths in a volume simultaneously during a scan by the TDI imager.

In any embodiments, one or more of the following features may be implemented in any combination and without limitation. The TDI imager may be tilted at an angle relative to the biological tissue sample such that focal planes for the plurality of partitions correspond to the plurality of different depths in the biological tissue sample. The plurality of partitions on the TDI imager may be physically separated by spaces between the plurality of partitions. The plurality of partitions on the TDI imager may be separated by a row of pixels that are covered. The plurality of different depths in the biological tissue sample may include a plurality of different depth ranges in the biological tissue sample. A partition in the plurality of partitions on the TDI imager may correspond to a depth range in the plurality of different depth ranges, the partition including a plurality of pixel rows, and the each of the plurality of pixel rows corresponding to a different depth in the depth range. Data received from the plurality of pixel rows may be combined in a focus-drilling combination to produce an image for the depth range. Data received from the plurality of pixel rows may be combined in a focus-drilling combination to produce an image for the depth range. The depth range may be between about 250 nm and about 750 nm. The biological tissue sample may be between about 2 μm and about 10 μm thick. Images of the biological tissue sample may be generated from each of the plurality of partitions. The TDI imager may be tilted at an angle relative to the volume such that focal planes for the plurality of partitions correspond to the plurality of different depths in the volume, and the angle may be adjustable to fine-tune the plurality of different depths in the volume. The system may include a glass cover on the TDI, where the glass cover may include a plurality of sections corresponding to the plurality of partitions, and thicknesses of the plurality of sections of the glass cover may cause the focal planes for the plurality of partitions be at the plurality of different depths in the volume. The system may include a lens in front of the TDI, where the lens may include a plurality of sections corresponding to the plurality of partitions, and thicknesses of the plurality of sections of the lens cause the focal planes for the plurality of partitions be at the plurality of different depths in the volume. The plurality of partitions of the TDI imager may have different heights relative to each other, and the different heights may cause the focal planes for the plurality of partitions be at the plurality of different depths in the volume. The volume may include a biological tissue sample. The system may include a lens in front of the TDI, where the lens may include a wedge shape, and the wedge shape may cause the focal planes for the plurality of partitions be at the plurality of different depths in the volume.

### BRIEF DESCRIPTION OF THE DRAWINGS

A further understanding of the nature and advantages of various embodiments may be realized by reference to the remaining portions of the specification and the drawings, wherein like reference numerals are used throughout the several drawings to refer to similar components. In some instances, a sub-label is associated with a reference numeral to denote one of multiple similar components. When reference is made to a reference numeral without specification to an existing sub-label, it is intended to refer to all such multiple similar components.

FIG. 1 illustrates a high-resolution biological imaging system, according to some embodiments.

FIG. 2 illustrates a flowchart of a process for capturing spatial images of a sample, according to some embodiments.

FIG. 3 illustrates how the imaging system may capture a plurality of images illuminated by different wavelengths, according to some embodiments.

FIG. 4 illustrates a TDI camera that may be used in the imaging system, according to some embodiments.

FIG. 5 illustrates a TDI imager with rows of imaging pixels divided into a plurality of partitions, according to some embodiments.

FIG. 6 illustrates a configuration for a TDI imager to simultaneously capture multiple image slices in the tissue sample during a single scan, according to some embodiments.

FIG. 7 illustrates a magnified view of a single partition with a corresponding focal plane in an image slice, according to some embodiments.

FIG. 8 illustrates a TDI imager with a stepped profile, according to some embodiments.

FIG. 9 illustrates how the TDI imager may be configured to capture image slices at multiple depths using a cover or lens having sections of varying thicknesses, according to some embodiments.

FIG. 10 illustrates a lens or glass cover having a wedge shape, according to some embodiments.

FIG. 11 illustrates a flowchart of a method of capturing spatial images of a biological tissue sample, according to some embodiments.

FIG. 12 illustrates an exemplary computer system, in which various embodiments may be implemented.

### DETAILED DESCRIPTION

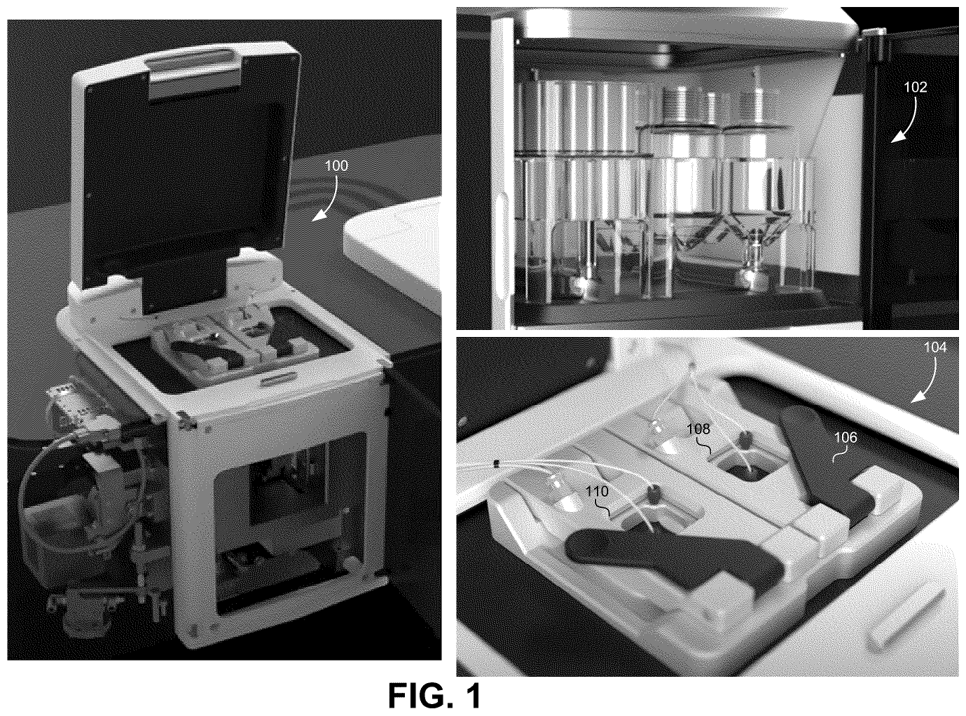

FIG. 1 illustrates a high-resolution biological imaging system 100, according to some embodiments. The imaging system 100 may be configured to combine multiple imaging workflows together into a single process to perform an automated spatial analysis of the tissue sample. The imaging system 100 may include multiple imaging chambers 108, 110, each of which may be configured to perform individual imaging operations on different tissue samples. A fluid system 102 may provide integrated fluid control to provide a plurality of fluorophores and/or other fluids to the imaging chambers 108, 110 during the imaging process. Different fluorophores and reagents may be loaded into containers in the fluid system 102 such that these fluids can be automatically provided to the imaging chambers 108, 110 when needed during the imaging process. In the context of this disclosure, the fluorophores may be attached to one or more binding reagents that specifically interact with one or more analytes in the tissue sample. Exemplary binding reagents include nucleic acid probes, proteins (such as antibodies and antibody derivatives), and aptamers. Thus, where fluorophores are mentioned in this disclosure, it is to be understood that the fluorophores may be present as a component of or attached to one or more binding reagents.

The imaging system 100 may include a computer system comprising one or more processors, one or more memory devices, and instructions stored on the one or more memory devices that cause the imaging system 100 to perform the imaging operations on the tissue samples in the imaging chambers 108, 110. Thus, each of the operations of the imaging process described herein may be represented by instructions stored on the one or more memory devices.

In an example imaging workflow, a user or automated process may load a tissue sample onto a slide, and load the slide into an imaging chamber 108. After securing the tissue sample in the imaging chamber 108, fluids may then be automatically pumped into the imaging chamber 108. For example, some fluids may be pumped into the imaging chamber 108 in order to clean the tissue and/or remove previous fluids or fluorophores that may be present in the imaging chamber 108. New fluids or fluorophores may be provided from the fluid system 102 in an automated fashion, as specified by the instructions executed by the controller. Generally, these “fluids” may more specifically include stains, probes, and other biological labels. During a typical cycle, one or more fluorophores may be pumped into the imaging chamber 108 that are configured to attach to the cells in the tissue sample in order to visually highlight different features within the sample. Corresponding laser wavelengths may then be used to illuminate the sample in the imaging chamber 108 to excite the fluorophores, and a camera may capture images of the illuminated sample. The fluorophores may be matched with different laser wavelengths that are configured to illuminate those specific fluorophores.

After the imaging process is complete, the raw images from the system may be converted into RNA spots or protein spots by the controller. These RNA spots or protein spots may be visualized as cell-type clusters that are highlighted by the different fluorophores. Multiple images may then be merged for a multi-omic analysis of the tissue sample. Software tools provided by the controller of the imaging system 100 may provide different visualizations, data filtering, and analysis tools for viewing the images.

Although the imaging system 100 is described herein as a fully integrated solution, combining control processing, image capture, and fluidics into a single integrated system, other embodiments may use systems that are distributed to some degree. As the imaging speed is increased using the techniques described below, it may become more advantageous to separate portions of the integrated system into distributed subsystems. For example, the fluid operations and the imaging operations need not be integrated into a single integrated tool. Multiple fluid chambers may be connected to a singular, stand-alone imaging tool using a robot or human that transfers material back and forth between the two. Therefore, the term “imaging system” should be construed broadly to encompass both fully integrated and distributed systems.

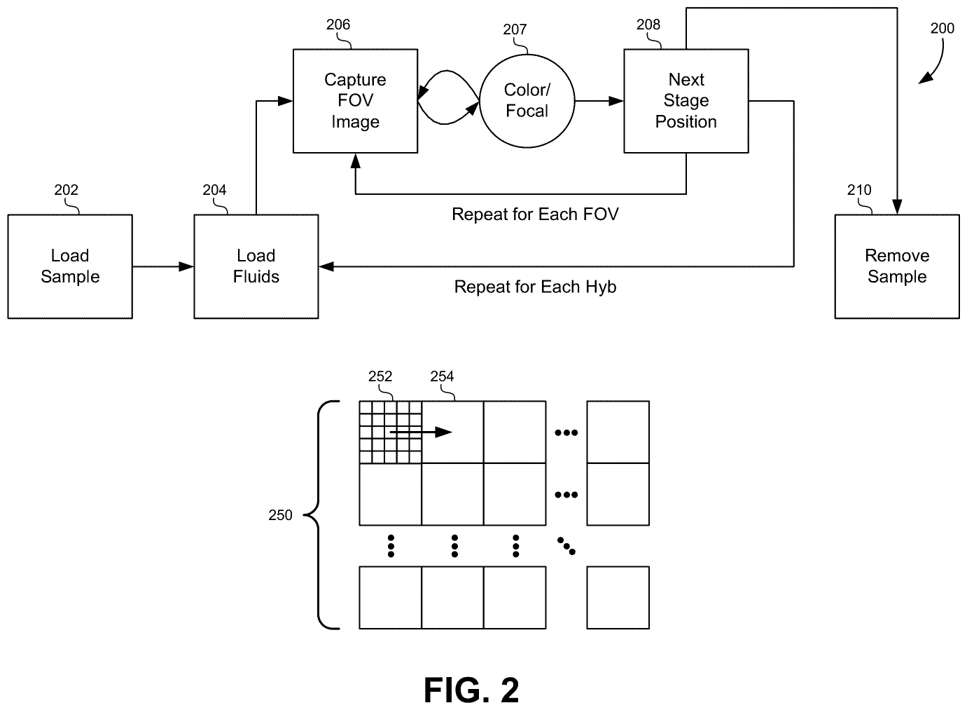

FIG. 2 illustrates a flowchart 200 of a process for capturing multi-omic images of a sample, according to some embodiments. As described above, the process may include loading a tissue sample on a substrate, such as a coverslip or a slide, and securing the tissue sample inside one of the imaging chambers of the imaging system (202). Note that multiple stations in the image processes system 100 may operate independently and simultaneously. For example, the imaging chamber 108 may capture images of the sample while another station may exchange fluids with a tissue sample. Some embodiments may also include a photo-bleaching station. The imaging system 100 may then provide fluids from the fluid system 102 into the imaging chamber 108 (204). These fluids may include fluorophores that are configured to attach to specific cell or tissue types that are to be highlighted in the resulting image.

In order to capture images with high resolution sufficient to visualize individual cells in detail, the image of the sample may be captured in stages. For example, instead of capturing a single image of the sample, the field-of-view of the camera in the imaging system 100 may be reduced to increase the resolution. Multiple images may then be captured of the sample and stitched together to form an overall image. For example, the overall image 250 may be comprised of multiple sub images that may be captured by the camera at a high resolution. Each of the images may correspond to a field-of-view of the image. Thus, the process may include incrementally capturing a field-of-view image using the camera (206), then moving the camera view to a subsequent location with an adjacent field-of-view and preparing the camera for the subsequent stage (208). At each field-of-view location, the process may iterate to capture images at different focal planes and/or with different light wavelengths (207), thus capturing multiple images at each position. This process may be repeated until the overall image 250 of the sample has been captured by the individual field-of-view images.

In order to capture the overall image 250, the field-of-view of the camera may move in a pattern over the tissue sample. For example, a first field-of-view 252 may be captured (206), then the camera may move to a second field-of-view 254 that is optionally sequential and/or adjacent to the first field-of-view 252 in a grid pattern (208). This process may be repeatedly executed for each field-of-view in the sample until the overall image 250 has been captured. Note that the grid pattern illustrated in FIG. 2 is provided only by way of example and is not meant to be limiting. Other embodiments may move horizontally, vertically, diagonally, and/or in any other pattern that may be used to capture individual field-of-view images that may be combined into the overall image 250. The individual fields-of-view may overlap in some embodiments, or may not overlap in other embodiments.

Multiple overall images 250 of the tissue sample may be captured in order to highlight different features in the tissue sample for the overall multi-omic analysis. Therefore, after the overall image 250 is captured for a particular fluorophore or set of fluorophores, the process may be repeated on the same tissue sample with another fluorophore or set of fluorophores. For example, the previous fluorophores may be pumped out of the imaging chamber 108, cleaning or rinsing agents may optionally be pumped through the imaging chamber 108 to clean the tissue sample, and a new set of fluorophores may be pumped into the imaging chamber 108 for the next image (204). Each overall image captured with different fluorophores to be combined in the multi-omic analysis may be referred to as an “imaging cycle” or a “hyb,” which is short for “hybridization,” in a “fluorophore labelling hybridization cycle.” Typically, each sample may be subject to a plurality of hybs using different fluorophores. For example, some embodiments may capture two, three, four, five, six, or more overall images of the sample corresponding to the number of unique fluorophores), thereby repeating the cycle (204) multiple times. When the desired number of images of the sample have been captured, the sample may be removed from the imaging chamber 108 (210). A new sample may then be added to the imaging chamber 108 (202), and the imaging process may be repeated.

At each field-of-view image location, the sample may be illuminated by a plurality of different light wavelengths (e.g., different colors configured to illuminate different fluorophores in the sample), and thus multiple images may be captured at different wavelengths at each location. Additionally, the sample itself may be adjusted axially to capture multiple images at different Z-depth levels, resulting in three-dimensional image slices through the tissue sample. As used herein, the term Z-depth may refer to a distance along a focal line of the camera, which may in some instances may also be perpendicular to the surface of the tissue sample. The tissue samples under analysis are three-dimensional volumes at different Z-depths in a layer of cells (i.e., different distances from the camera or lens within the volume of the tissue sample). Therefore, in order to capture a three-dimensional representation of the tissue sample, the imaging system 100 may capture complete images at different Z-depths by adjusting the focal length of the camera. For example, some embodiments may slice the volume of the tissue sample at 0.5 μm intervals (i.e., images are taken at or about −1.0 μm, −0.5 μm, 0.0 μm, 0.5 μm, and 1.0 μm along the Z-axis). This range, for example, may represent slices all within one layer of cells, where a cell may be about 10 μm to about 30 μm thick. While this process does provide high-resolution, multi-omic image data, this process also takes a considerable amount of time. For example, when capturing images at seven different Z-depths with four different fluorophores, 28 scans through the tissue sample may be used. Each movement from one field-of-view to the next field-of-view include significant overhead that increases the time required to capture each image. The process may include moving the sample laterally such that the camera captures a new field-of-view location, which may require time for acquiring the new images, physically moving the sample using a piezo motor or stage motor, configuring the laser and the corresponding filter for the desired wavelength, stabilizing and focusing the camera, and/or other operations. Combining these different factors together causes the overall imaging time to be relatively large. For example, each hyb may take approximately 10 hours to complete in an example implementation using a camera with a 40× objective and a 30×30 grid of field-of-view images to cover the sample. A typical four-hyb session may then take between 30-40 hours total to complete. While reducing the resolution of the camera increases the field-of-view and reduces the total number of field view images required, this also negatively affects the quality of the resulting images. This significant time requirement represents a technical problem in the area of biological spatial omics.

Some embodiments may reduce the overhead of moving the complete field-of-view for the imaging camera and instead use a Time Delay Integration (TDI) camera. The TDI camera may be used to continuously scan the tissue sample in columns rather than moving between different fields of view. The laser beam that is projected onto the imaging sample may be shaped to approximately match the TDI image scan line. Switching to a TDI camera may improve many of the sources of error and overhead challenges listed above. TDI scanning enables a continuous scanning image collection which averages many non-uniformities in the scan direction. This reduces the system sensitivity to many different error sources including illumination non-uniformity, image sensor pixel-to-pixel non-uniformity (and defects), and/or lens aberrations. TDI scans stitch on two sides instead of on four sides, and scanning under constant acceleration may reduce acceleration force ripples that cause vibrations in the tissue sample. Finally, overhead from mechanical movements may be greatly reduced, whereas a system with 100 fields (10'10 square), 4-color, 5-focus will require only 195 over-head events [e.g., (9 scans×4 color+3 color changes)×5 focus].

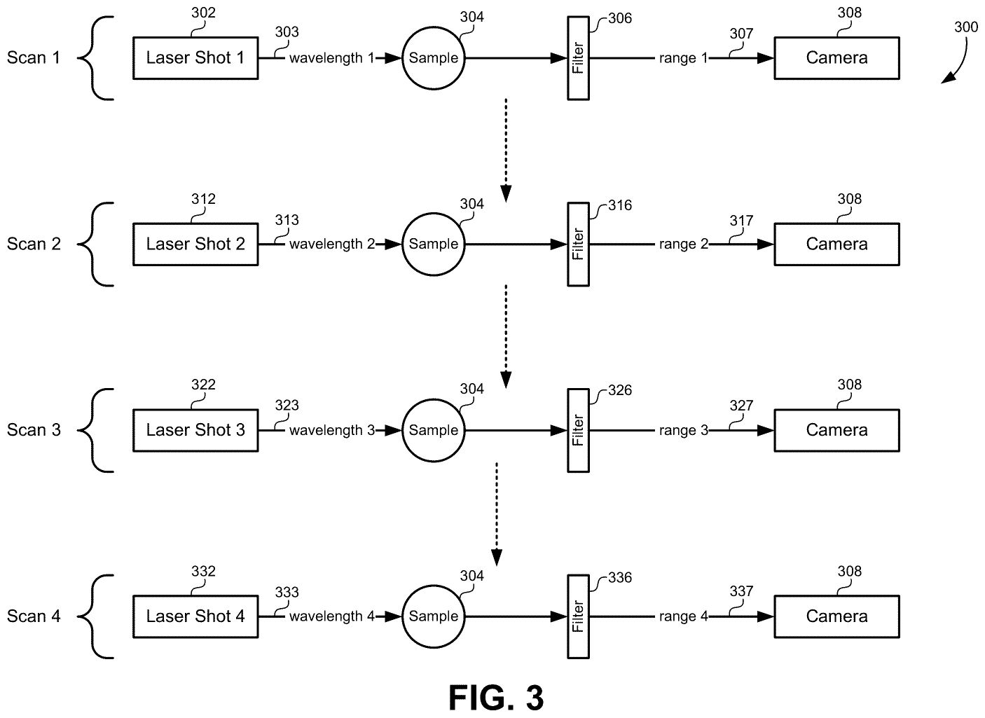

FIG. 3 illustrates how the imaging system 100 may capture a plurality of images illuminated by different wavelengths, according to some embodiments. In this example, each area of the tissue sample may be illuminated by four different wavelengths. A first laser shot 302 may illuminate an area of the tissue sample 304 with a first wavelength 303. Reflected light or fluorescence from the first laser shot 302 may pass through a first filter 306 configured to pass a first wavelength range 307 before being recorded by the camera 308. Note that the first wavelength range 307 may be slightly higher than the first wavelength 303 from the first laser shot 302 and thus the excitation wavelength of the first laser shot 302 may be blocked by the first filter 306. For example, a fluorophore may be activated at a given wavelength, and the activated fluorophore may emit light within a wavelength range that is greater than the activation wavelength. Similarly, a second laser shot 312 may illuminate the area of the tissue sample 304 with a second wavelength 313. Reflected light or fluorescence from the second laser shot 312 may pass through a second filter 316 configured to pass a second wavelength range 317 before being recorded by the camera 308. A third laser shot 322 may illuminate the area of the tissue sample 304 with a third wavelength 323. Reflected light or fluorescence from the third laser shot 322 may pass through a third filter 326 configured to pass a third wavelength range 327 before being recorded by the camera 308. A fourth laser shot 332 may illuminate the area of the tissue sample 304 with a fourth wavelength 333. Reflected light or fluorescence from the fourth laser shot 332 may pass through a fourth filter 336 configured to pass a fourth wavelength range 337 before being recorded by the camera 308. The images captured by the camera 308 by each laser shot may be stitched together to form for complete images of the tissue sample, each illuminated by a different wavelength.

Typically, a complete set of field-of-view images may be captured for one each wavelength at each field-of-view location before moving to the next location. Between capturing images at each wavelength, time is required to change the filter wheel, settle the filter wheel, move the motor to account for wavelength-dependent focal plan shifts, and so forth. Therefore, each additional desired wavelength increases the total time for imaging a tissue sample.

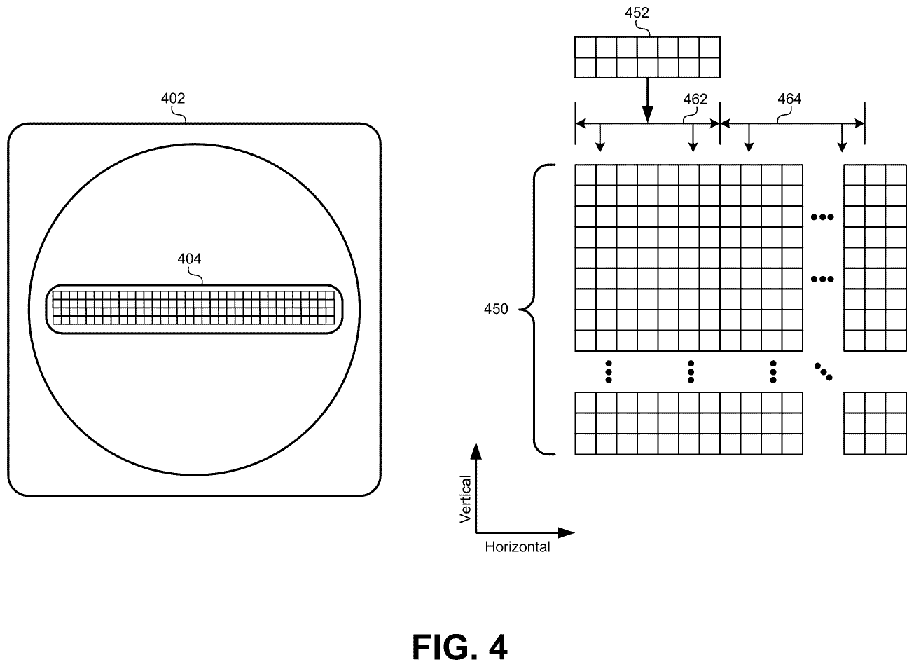

FIG. 4 illustrates a TDI camera 402 that may be used in the imaging system, according to some embodiments. The TDI camera 402 may include a charge-coupled device (CCD) or a CMOS photon-detecting device as an image sensor for capturing images. For example, the TDI camera 402 may include a scan line 404 of individual CCD pixels in a horizontal configuration as depicted in FIG. 4. Note that only a single partition of pixels is illustrated in this scan line 404 for the sake of clarity. However, this is not meant to be limiting. As discussed and shown below, TDI cameras 402 may include multiple horizontal rows of pixels organized into one or more partitions.

The operation of the TDI camera 402 may be contrasted with the operation of the traditional camera described above. As described above, traditional cameras may capture a single field-of-view, and then move to another, nonoverlapping field-of-view before capturing the next image. Turning back briefly to FIG. 2, a field-of-view 252 may include a horizontal grid of individual pixels within the field-of-view 252, and each of the individual pixels will capture the image simultaneously when the camera shot is acquired. In contrast, the TDI camera 402 may use the scan line 404 of individual pixel rows. The TDI camera 402 may continuously scan in the vertical direction over the image sequentially. The movement of the tissue sample and/or TDI camera 402 may be synchronized such that images are captured at each pixel step. The last horizontal line of pixels in the scan line 404 may accumulate and average the individual pixels to output an average reading for that scan location. The whole image may then be assembled from the equally spaced lines through the linear field-of-view of the scan line 404. Note that the terms “horizontal” and “vertical” are used merely to denote orthogonal directions as illustrated in FIG. 4 and are not meant to be limiting to a specific direction.

The scan line 404 need not extend the entire horizontal length of the image. Instead, multiple vertical “columns” may be captured using multiple vertical continuous scans. For example, to capture an overall image 450, a scan line 452 may continuously scan down a first vertical column 462 of the imaging area. When the scan of the first vertical column 462 is completed, the scan line 452 of the TDI camera may be repositioned over a second vertical column 464, and the scan line 452 may then continuously scan down the second vertical column 464. These vertical columns may be stitched together to form the overall image 450 of the tissue sample.

Use of the TDI camera 402 represents a significant technical improvement over other cameras in scanning tissue samples. The TDI camera 402 may continuously capture each vertical scan column, which eliminates the need to mechanically reposition the sample, stabilize, focus, and prepare for each individual field-of-view capture. Instead, the TDI camera 402 may move at a constant speed in the vertical direction and scan continuously to accumulate the reflected light or fluorescence signals from the tissue sample. The only repositioning that needs to occur for the TDI camera 402 may be in between each of the vertical column captures.

As described above, generating a full volumetric image of a biological tissue sample not only typically uses iterative imaging of the sample with different light source and color filter combinations, but it also uses imaging at multiple focal planes within the volume of the biological tissue sample. This generates images at multiple focal planes to produce a multi-slice volumetric image, much like a Plenoptic Camera, a light-field camera, or a 3-D Confocal Microscope. Ideally, each individual slice should image all of the fluorophores over a depth range within an volumetric image slice of the sample, while not imaging fluorophores in neighboring volumetric image slices. For example, typical biological tissue samples may be between approximately 2 μm and approximately 10 μm thick. Imaging slices through the volume may be taken at regular intervals, such as every 0.5 μm (e.g., images may be recorded at −1.0 μm, −0.5 μm, 0.0 μm, 0.5 μm, 1.0 μm, .etc.). Some embodiments may capture an image at a specific Z-depth within these depth ranges, while other embodiments may capture an image that represents the average of incremental depths throughout the depth range in each of these image slices. These embodiments are described in detail below.

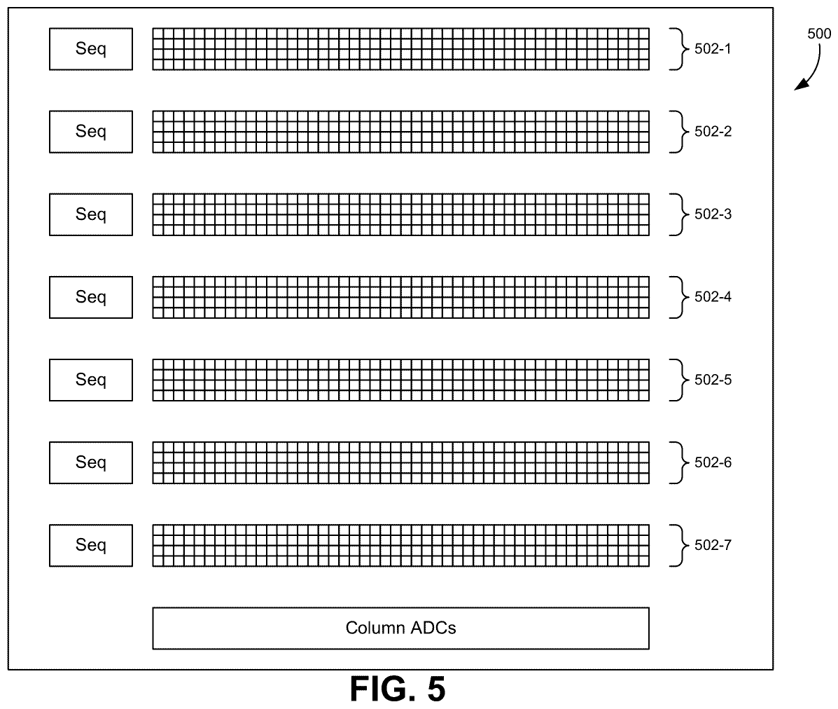

FIG. 5 illustrates a TDI imager 500 with rows of imaging pixels divided into a plurality of partitions, according to some embodiments. Conventional TDI imaging would scan the entire biological sample n times in order to obtain n images at n focus depth locations found within n volumetric slices. For example, when dividing the tissue sample into five volumetric slices, the TDI imager would complete one complete scan five times, with each scan using a different focal plane for the TDI imager, repeated for each fluorophore color. These focal planes would land around the center of each of the volumetric image slices. The TDI imager 500 illustrated in FIG. 5 divides the horizontal pixel rows into a plurality of partitions 502. The horizontal pixel rows within each of the plurality of partitions 502 may function independently as separate TDI sub-imagers. In some commercial implementations, the TDI imager 500 may include different color filters placed over each of the partitions 502. For example, some existing TDI imagers may include a 3-band RGB color-segmented set of TDI partitions, with each partition configured to capture a different wavelength range. In the example of FIG. 5, the TDI imager may include seven partitions 502, each of which may independently capture a view of a scanned image.

Prior to this disclosure, conventional uses of a multi-partition TDI imager, such as the TDI imager 500 illustrated in FIG. 5, have been used primarily to capture different light wavelengths of a view that is common to all of the partitions 502. In other words, each of the partitions 502 were configured to capture images at a common focal plane. While the color filters may be different for each of the partitions 502, the focal plane of the image being received in each partition was the same.

The embodiments described herein may configure the TDI imager 500 in order to capture images at different focal planes that correspond to different volumetric depth ranges or image slices in the tissue sample. By independently moving the focal planes of each of the partitions 502 of the TDI imager, each of the partitions 502 may be configured to capture a different image slice within the tissue sample. The TDI imager 500 may then simultaneously scan images at different depths slices within the volume of the tissue sample. For example, instead of requiring seven separate and complete scans of the tissue sample to acquire images at seven different image slices in the volume of the sample, a single scan of the sample may capture images at each of the seven image slice depths simultaneously. This represents a significant improvement in the total time required to image a tissue sample. As described above, capturing images at seven different volumetric depths using four different fluorophore combinations previously required 28 complete scans through the tissue sample. When configuring the partitions 502 of the TDI imager 500 to simultaneously capture all of the image slice depths during a single scan, the total number of scans may be reduced from 28 down to four.

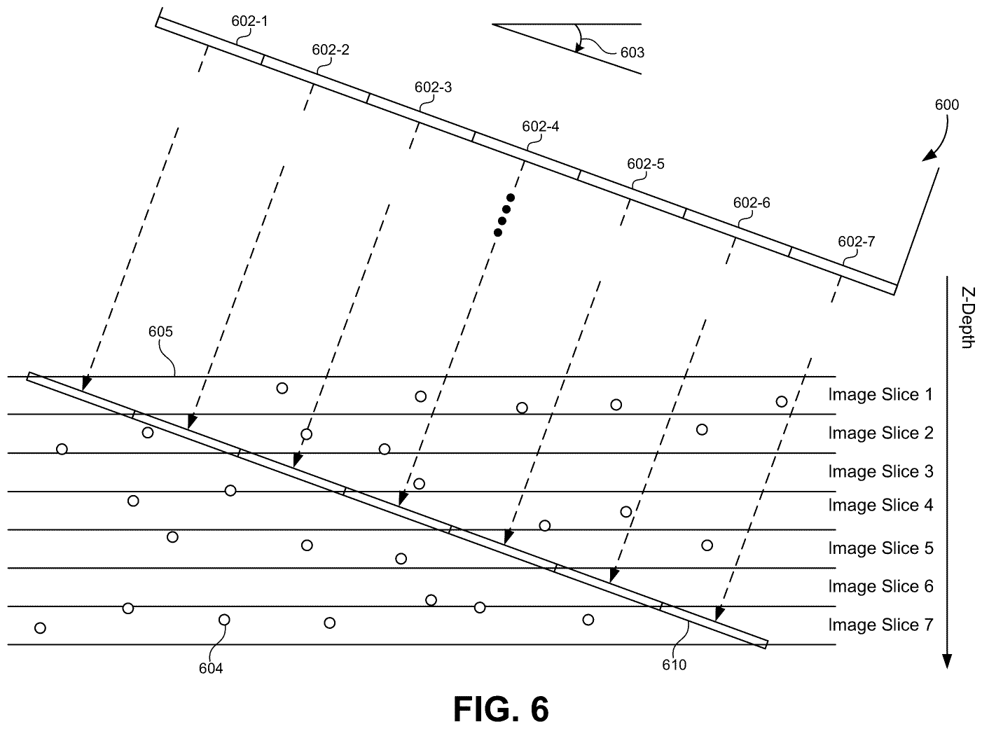

FIG. 6 illustrates a configuration for a TDI imager 600 to simultaneously capture

multiple image slices in the tissue sample during a single scan, according to some embodiments. One method of configuring the TDI imager 600 is to position the TDI imager 600 at a tilt angle 603 relative to the surface of the tissue sample 605. By tilting the TDI imager 600, the focal planes 610 of each partition 602 may also be angled such that they penetrate different depths into the tissue sample 605. By controlling the tilt angle 603, the corresponding focal planes 610 for each of the partitions 602 for the TDI imager 600 may be aligned with the boundaries of the different desired image slices within the volume of the tissue sample. The tilt angle 603 may thus be selected based on the thickness of the tissue sample 605, the total number of desired image slices, and/or the total number of partitions 602. Alternatively, some embodiments may install the TDI sensor normally (i.e., in a flat position, orthogonal to the optical axis) and then tilt the lens.

By properly aligning the tilt angle 603, each of the partitions 602 may be configured to image all of the volume within the corresponding image slice, while effectively excluding imaging portions of the volume that are outside of the corresponding image slice. As depicted in FIG. 6, the field of view for each of the partitions 602 may be aligned with the boundaries of the image slices in the tissue sample 605 to control the depth range imaged by each partition 602. By tilting the focal plane as shown in FIG. 6, each partition 602 of the TDI imager 600 may completely image one of the different corresponding volumetric slices without overlapping a neighboring image slice. Note that no changes to the architecture of the TDI imager 600 are required by this implementation. Instead, a commercial multi-partition TDI imager 600 or the lens may be tilted within the assembly of the imaging system according to the tilt angle 603 to generate the multi-depth image capture capability.

In addition to initially aligning the tilt angle 603, some embodiments may allow the placement of the focal planes 610 to be fine-tuned by adjusting the tilt angle 603. For example, software/hardware controls may be provided that allow the tilt angle 603 of the TDI imager and/or the lens to be adjusted in order to move the focal planes 610 in the sample. This ability to find-tune the tilt angle 603 provides an advantage for this implementation over other implementations.

Note that this disclosure uses a number of partitions and image slices, such as five partitions or seven partitions, only by way of example. These partition and image slice examples are not meant to be limiting. Other embodiments may use more or fewer partitions and/or image slices without limitation. For example, some embodiments may use three partitions in the TDI imager corresponding to three image slices in the tissue. Other embodiments may use two partitions, four partitions, six partitions, eight partitions, nine partitions, 10 partitions, or more, each with a corresponding number of image slices in the tissue. Also note that the following figures may omit the lens itself from the diagrams for the sake of clarity. However, it should be understood that a lens would be placed in between the TDI imager and the tissue sample in actual implementations. Furthermore, the dotted lines from the TDI partitions to the image slices in the tissue sample do not represent rigorous ray tracing, since the lens would alter these optical paths.

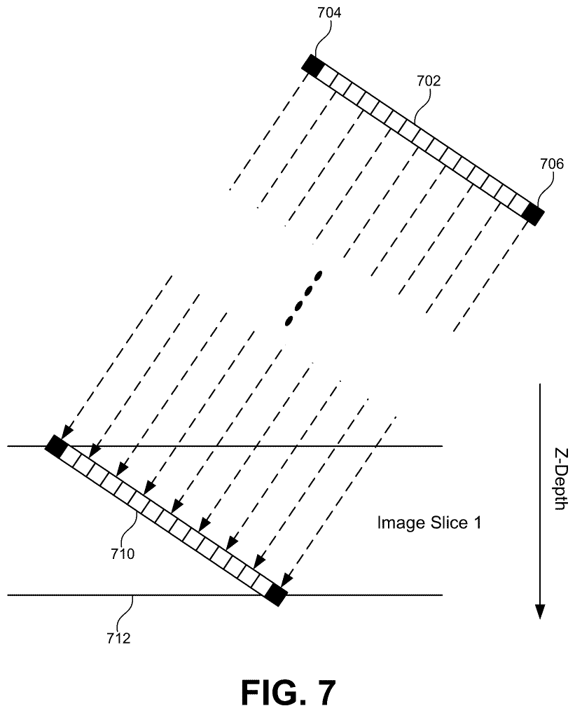

FIG. 7 illustrates a magnified view of a single partition 702 with a corresponding focal plane 710 in an image slice 712, according to some embodiments. This view illustrates how the single partition 702 may be made up of a plurality of individual horizontal pixel rows. As a cross-sectional view of the single partition 702, this view shows a cross section of individual pixel rows within the partition 702. Note that 17 pixel rows is used only by way of example in FIG. 7 and is not meant to be limiting. Embodiments of the TDI imager may include partitions with any number of pixel rows, such as 32 rows, 64 rows, 128 rows, 256 rows, and so forth.

Each of the individual pixel rows in the partition 702 has a focal plane that corresponds to a different Z-depth level within the image slice 712. Therefore, a single partition of the TDI imager may scan through an image slice 712 at progressively sequential depths, even though movement of the tissue sample relative to the TDI camera is parallel. Instead of negatively affecting the imaging, this configuration has been shown to enhance the ability to accurately image the entire image slice. For example, if the focal plane for all of the horizontal pixel rows in the partition 702 have the same focal plane, the resulting image may miss some fluorophores that are within the image slice 712 but not precisely at the depth of the focal plane for the partition 702. However, by angling the TDI imager, and consequently angling each of the pixel rows within each of the partitions, pixels may be aligned at many different focal planes within each of the image slices, thus capturing fluorophores that occur anywhere in the depth range of the image slice. This provides a more complete and accurate view of the volume of the tissue sample.

The partition 702 may continue to function as a traditional TDI imager, where the signal received from a previous row may be aggregated with a signal received from a current row, etc., as the imager or tissue sample moves. An aggregated image generated by the partition 702 would therefore aggregate signals throughout the depth range of the image slice 712 to generate a single image that represents the depth range of the whole image slice 712. This combination of individual pixel rows at different depths is referred to herein as “focus drilling.” In the example described above, the slicing interval (0.5 μm) is approximately equal to the anticipated nominal focus drilling amplitude (0.5 μm), which enables a multi-partition TDI sensor to capture multiple image slices in a single scan, with each slice being combined in a focus-drilling combination to more uniformly capture all the fluorophores within each slice. The seven different volumetric slices corresponding to the seven different partitions on the TDI sensor may each be focus drilled to more effectively capture of all the fluorophores within that slice. All seven volumetric slices may also be captured in a single scan.

FIG. 7 also illustrates how an image from one image slice 712 may be separated from images generated by neighboring image slices. First, using a focus-drilling combination of the rows at different focal planes within the partition generates a process window with a steep fall-off slope. Instead of a low-slope or gradually decaying slope at the edge of the process window, the focus-drilling combination develops a process window having more of a “top hat” shape with an adjustable width (i.e., adjustable for the image slice thickness in the Z-depth direction), depending on the degree of focus drilling. In practice, this steep fall-off at the edges of the processing window tend to isolate one image slice from another, with a large stable region within the processing window for capturing the volumetric image. This generates more discrete volumetric image slices with a very controllable thickness compared to previous techniques.

When a fluorophore occurs on a boundary between two image slices, this may result in duplicating the image of the fluorophore in both of the neighboring image slices. If the unwanted duplicate imaging of a fluorophore in a neighboring volumetric slice creates a significate problem, this can be mitigated by having a small separation between the partitions on the TDI imager. This can either be performed by having the partitions designed with a physical separation between them, or if working with an OEM sensor by having a black-matrix resist patterned over the array that blocks certain rows in the TDI sensor. This partition separation approach would decrease the focus drilling amplitude to less than the volumetric slicing pitch and guard-band against duplicate fluorophore imaging in a neighboring image slice.

For example, some configurations may isolate one image slice from neighboring image slices using a physical separation or space. In the example of the TDI imager illustrated in FIG. 5, the partitions 502 on the TDI imager 500 are physically separated by spaces between the partitions 502. For example, the horizontal pixel rows within each partition may be closely spaced next to each other, while the spacing between the horizontal pixel rows in adjacent partitions may be much greater than the row spacing within the partitions (e.g., 5 times greater, 10 times greater, etc.).

As an alternative to physically separating the partitions, partitions may be created in a TDI imager array by covering one or more rows of horizontal pixels in order to generate partitions. For example, a dark photoresist may be placed over one or more pixel rows in order to separate the rows from each other and create partitions in the TDI imager array. For example, FIG. 7 illustrates pixel 704 and pixel 706 at the edge of the partition 702 that have been darkened, blocked, or otherwise obscured or omitted from the partition 702 in order to isolate the image from the neighboring image slices.

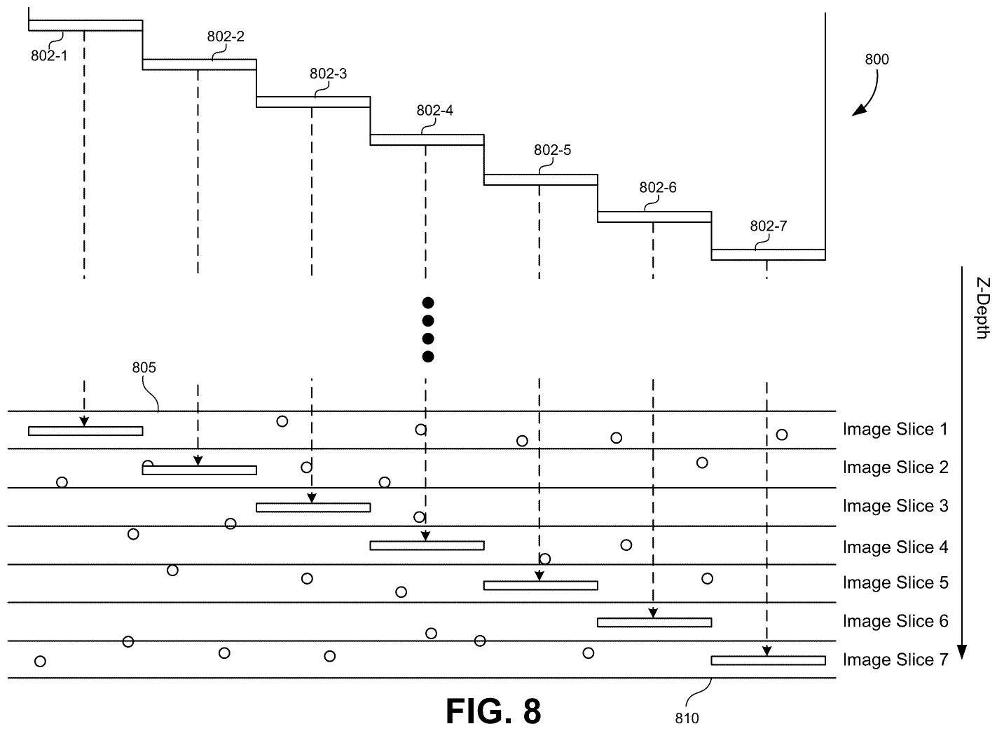

FIG. 8 illustrates a TDI imager 800 with a stepped profile, according to some embodiments. Instead of configuring the TDI imager to be tilted at an angle relative to the volume such that the focal planes for the partitions correspond to different depths in the volume, other embodiments may use alternate configurations of the TDI imager or other system components to generate a similar effect. For example, FIG. 8 uses a TDI imager where each of the plurality of partitions 802 have different heights relative to each other in the device itself. Steps of filler material may be formed on the substrate of the imager beneath the imaging pixels in order to adjust the relative height between these partitions. The height difference between the partitions 802 may correspond to the relative thickness of the image slices. Each of the partitions may then correspond to a different depth range in the different image slices.

In contrast to the configuration with the tilted TDI imager, the stepped TDI imager may generate a focal plane for each of the horizontal pixel rows within each partition at the same Z-depth in the tissue sample. Therefore, focus-drilling combinations of the pixel rows need not be used, and these implementations may instead generate an image with a focal plane concentrated at one location within the depth range of the image slice as illustrated in FIG. 8. These implementations may use custom TDI imagers that are constructed specifically for tissue samples having a known or predictable thickness. As described below, the depth of the focal planes for each of the partitions 802 may be fine-tuned using sections of a glass cover over the imager or lens sections.

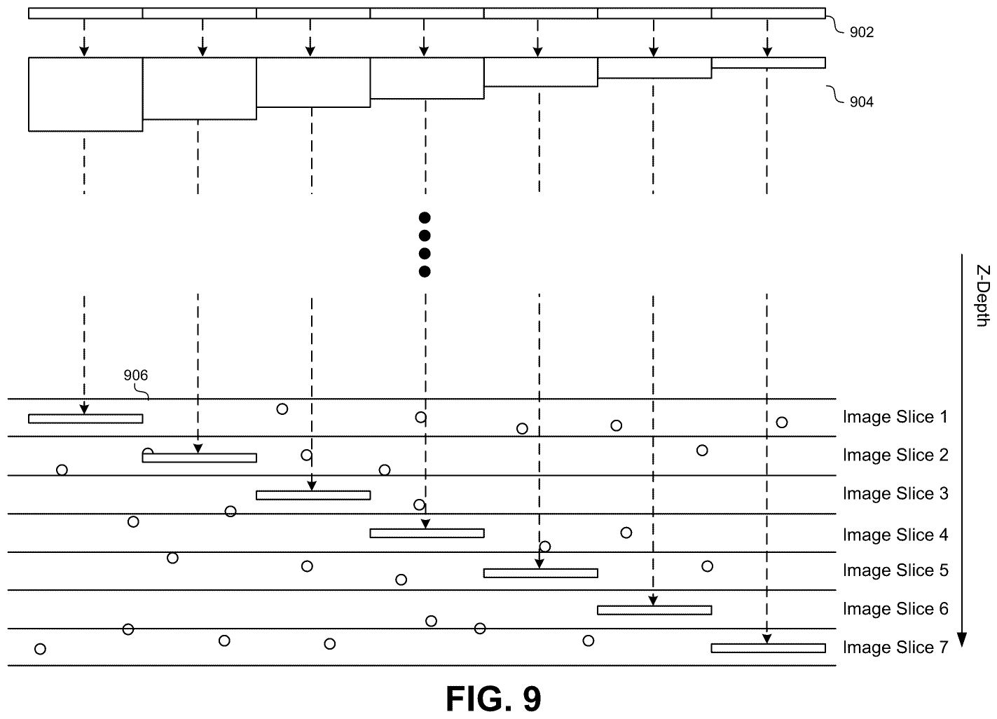

FIG. 9 illustrates how the TDI imager may be configured to capture image slices at multiple depths using a cover or lens having sections of varying thicknesses, according to some embodiments. Instead of adjusting the angle or profile of the TDI imager 902, the TDI imager 902 may be oriented parallel to the tissue sample 906, with each of the partitions in the TDI imager 902 at the same level relative to the tissue sample. Normally, this would generate focal planes at the same Z depth for each of the partitions, and would only allow image capture at a single image slice at a time. However, by incorporating a layer 904 in front of the TDI imager 902, the focal lengths from each partition may be adjusted to correspond to the different image slices in the tissue sample 906.

For example, the layer 904 may be implemented as a glass cover on the TDI imager 902. The glass cover may include a plurality of sections that correspond to the plurality of partitions on the TDI imager 902. The sections on the glass cover may vary in thickness and may be adjusted for each partition to center the corresponding focal plane of the underlying partition in the center of one of the image slices. Similarly, the layer 904 may be implemented as a lens in front of the TDI imager 902. The sections having varying thicknesses may be implemented in the lens to center the focal planes of the underlying partitions of the TDI sensor 902 within the various image slices.

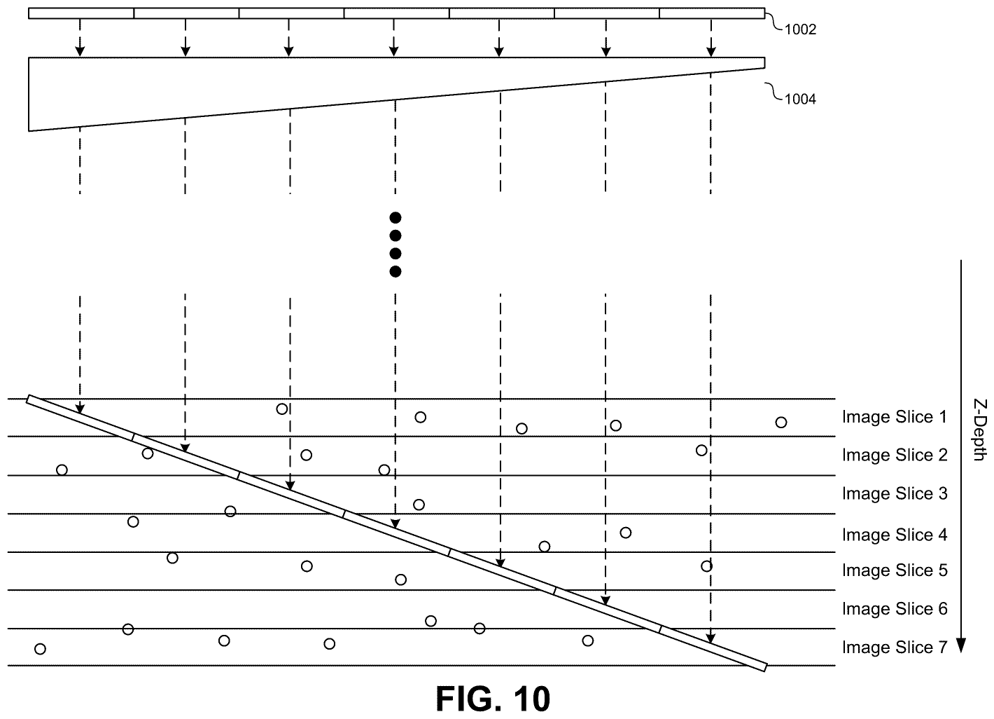

FIG. 10 illustrates a lens or glass cover having a wedge shape, according to some embodiments. Instead of using discrete, stepped sections having discrete thicknesses, a layer 1004 may be placed in front of the TDI imager 1002 having a continuous wedge shape. The layer 1004 may be implemented as a glass cover on the TDI imager 1002 and/or as a lens in front of the TDI imager 1002. Note that the wedge-shaped lens or cover generates a similar effect to tilting the TDI imager. Specifically, the focal planes for each partition step incrementally with each horizontal pixel row within the image slice. This allows the focus-drilling combination of the pixel rows described above to be implemented in this configuration. For example, the angle of the wedge-shaped lens or cover may be similar to the tilt angle 603 for the TDI sensor in FIG. 6.

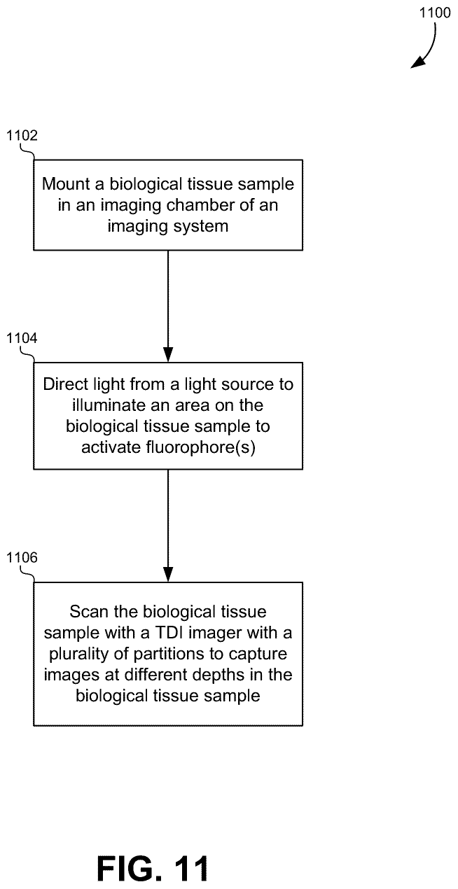

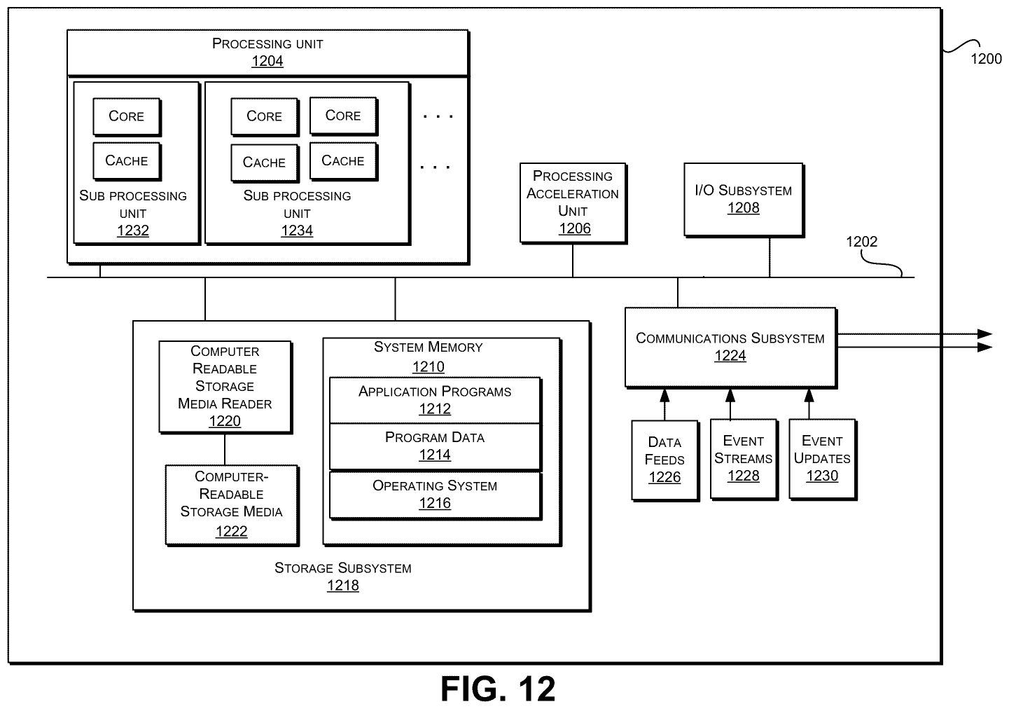

FIG. 11 illustrates a flowchart 1100 of a method of capturing spatial images of a biological tissue sample, according to some embodiments. This method may be executed by the imaging system 100 described above. For example, each of the steps in this method may be embodied in a set of instructions that is executed by a controller of the imaging system. The controller may include one or more processors that execute instructions stored on one or more memory devices to perform the operations described below. For example, the controller of the imaging system may be configured (or programmed) to cause the TDI imager to scan the biological tissue sample. An example of a computer system that may be used as a controller is described below in FIG. 12.

The method may include mounting a biological tissue sample in an imaging chamber of an imaging system (1102). The tissue sample may include any type of biological material, and may be mounted to a slide, coverslip, or other transparent surface. As described above, one or more fluorophores may be added to the imaging chamber to mix with the tissue sample.

The method may also include directing light from a light source to illuminate an area on the biological tissue sample to activate one or more fluorophores in the biological tissue sample (1104). As described above in FIG. 3, specific light wavelengths may be generated from a laser or other light source to activate specific fluorophores in the sample. In some embodiments, multiple wavelength may be provided at once, or multiple wavelength may be provided sequentially such the specific fluorophores may be highlighted in each image. Activated fluorophores may return light wavelengths that fall within a range above the activating wavelength for the corresponding fluorophore.

The method may further include scanning the biological tissue sample with a TDI imager having a plurality of partitions (1106). The plurality of partitions may be configured to capture images at a plurality of different depths in the biological tissue sample simultaneously during the scan. For example, the TDI imager may be positioned at a tilt angle relative to the tissue sample such that the focal planes of each partition fall within different depth ranges of the tissue sample. Alternatively, the TDI imager may be manufactured with a stepped profile, or a lens or glass cover may be used to shift the focal planes for each partition into the different image slices of the sample as described above in FIGS. 6-10. Some configurations may allow for a focus-drilling combination of the individual pixel rows within a partition as described above.

It should be appreciated that the specific steps illustrated in FIG. 10 provide particular methods of capturing spatial images of a biological tissue sample according to various embodiments. Other sequences of steps may also be performed according to alternative embodiments. For example, alternative embodiments may perform the steps outlined above in a different order. Moreover, the individual steps illustrated in FIG. 10 may include multiple sub-steps that may be performed in various sequences as appropriate to the individual step. Furthermore, additional steps may be added or removed depending on the particular applications. Many variations, modifications, and alternatives also fall within the scope of this disclosure.

The examples above recite a biological tissue sample as the volume being imaged by the imaging system. However, these techniques may also be expanded to other transparent volumes that do not necessarily include biological tissue. Specifically, each of the techniques described above may also be used to image any transparent volume at multiple focal planes during a single scan of a TDI imager.

FIG. 12 illustrates an exemplary computer system 1200, in which various embodiments may be implemented. The system 1200 may be used to implement any of the computer systems described above, including the controller of the imaging system 100. As shown in the figure, computer system 1200 includes a processing unit 1204 that communicates with a number of peripheral subsystems via a bus subsystem 1202. These peripheral subsystems may include a processing acceleration unit 1206, an I/O subsystem 1208, a storage subsystem 1218 and a communications subsystem 1224. Storage subsystem 1218 includes tangible computer-readable storage media 1222 and a system memory 1210.

Bus subsystem 1202 provides a mechanism for letting the various components and subsystems of computer system 1200 communicate with each other as intended. Although bus subsystem 1202 is shown schematically as a single bus, alternative embodiments of the bus subsystem may utilize multiple buses. Bus subsystem 1202 may be any of several types of bus structures including a memory bus or memory controller, a peripheral bus, and a local bus using any of a variety of bus architectures. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard.

Processing unit 1204, which can be implemented as one or more integrated circuits (e.g., a conventional microprocessor or microcontroller), controls the operation of computer system 1200. One or more processors may be included in processing unit 1204. These processors may include single core or multicore processors. In certain embodiments, processing unit 1204 may be implemented as one or more independent processing units 1232 and/or 1234 with single or multicore processors included in each processing unit. In other embodiments, processing unit 1204 may also be implemented as a quad-core processing unit formed by integrating two dual-core processors into a single chip.

In various embodiments, processing unit 1204 can execute a variety of programs in response to program code and can maintain multiple concurrently executing programs or processes. At any given time, some or all of the program code to be executed can be resident in processor(s) 1204 and/or in storage subsystem 1218. Through suitable programming, processor(s) 1204 can provide various functionalities described above. Computer system 1200 may additionally include a processing acceleration unit 1206, which can include a digital signal processor (DSP), a special-purpose processor, and/or the like.

I/O subsystem 1208 may include user interface input devices and user interface output devices. User interface input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. Additionally, user interface input devices may include voice recognition sensing devices that enable users to interact with voice recognition systems (e.g., Siri® navigator), through voice commands.

User interface input devices may also include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, medical ultrasonography devices. User interface input devices may also include, for example, audio input devices such as MIDI keyboards, digital musical instruments and the like.

User interface output devices may include a display subsystem, indicator lights, or non-visual displays such as audio output devices, etc. The display subsystem may be a cathode ray tube (CRT), a flat-panel device, such as that using a liquid crystal display (LCD) or plasma display, a projection device, a touch screen, and the like. In general, use of the term “output device” is intended to include all possible types of devices and mechanisms for outputting information from computer system 1200 to a user or other computer. For example, user interface output devices may include, without limitation, a variety of display devices that visually convey text, graphics and audio/video information such as monitors, printers, speakers, headphones, automotive navigation systems, plotters, voice output devices, and modems.

Computer system 1200 may comprise a storage subsystem 1218 that comprises software elements, shown as being currently located within a system memory 1210. System memory 1210 may store program instructions that are loadable and executable on processing unit 1204, as well as data generated during the execution of these programs.

Depending on the configuration and type of computer system 1200, system memory 1210 may be volatile (such as random access memory (RAM)) and/or non-volatile (such as read-only memory (ROM), flash memory, etc.) The RAM typically contains data and/or program modules that are immediately accessible to and/or presently being operated and executed by processing unit 1204. In some implementations, system memory 1210 may include multiple different types of memory, such as static random access memory (SRAM) or dynamic random access memory (DRAM). In some implementations, a basic input/output system (BIOS), containing the basic routines that help to transfer information between elements within computer system 1200, such as during start-up, may typically be stored in the ROM. By way of example, and not limitation, system memory 1210 also illustrates application programs 1212, which may include client applications, Web browsers, mid-tier applications, relational database management systems (RDBMS), etc., program data 1214, and an operating system 1216. By way of example, operating system 1216 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® 10 OS, and Palm® OS operating systems.

Storage subsystem 1018 may also provide a tangible computer-readable storage medium for storing the basic programming and data constructs that provide the functionality of some embodiments. Software (programs, code modules, instructions) that when executed by a processor provide the functionality described above may be stored in storage subsystem 1218. These software modules or instructions may be executed by processing unit 1204. Storage subsystem 1218 may also provide a repository for storing data used in accordance with some embodiments.

Storage subsystem 1200 may also include a computer-readable storage media reader 1220 that can further be connected to computer-readable storage media 1222. Together and, optionally, in combination with system memory 1210, computer-readable storage media 1222 may comprehensively represent remote, local, fixed, and/or removable storage devices plus storage media for temporarily and/or more permanently containing, storing, transmitting, and retrieving computer-readable information.

Computer-readable storage media 1222 containing code, or portions of code, can also include any appropriate media, including storage media and communication media, such as but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media. This can also include nontangible computer-readable media, such as data signals, data transmissions, or any other medium which can be used to transmit the desired information and which can be accessed by computing system 1200.

By way of example, computer-readable storage media 1222 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 1222 may include, but is not limited to, Zip® drives, flash memory cards, universal serial bus (USB) flash drives, secure digital (SD) cards, DVD disks, digital video tape, and the like. Computer-readable storage media 1222 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 1200.

Communications subsystem 1224 provides an interface to other computer systems and networks. Communications subsystem 1224 serves as an interface for receiving data from and transmitting data to other systems from computer system 1200. For example, communications subsystem 1224 may enable computer system 1200 to connect to one or more devices via the Internet. In some embodiments communications subsystem 1224 can include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 802.11 family standards, or other mobile communication technologies, or any combination thereof), global positioning system (GPS) receiver components, and/or other components. In some embodiments communications subsystem 1224 can provide wired network connectivity (e.g., Ethernet) in addition to or instead of a wireless interface.

In some embodiments, communications subsystem 1224 may also receive input communication in the form of structured and/or unstructured data feeds 1226, event streams 1228, event updates 1230, and the like on behalf of one or more users who may use computer system 1200.

By way of example, communications subsystem 1224 may be configured to receive data feeds 1226 in real-time from users of social networks and/or other communication services such as Twitter® feeds, Facebook® updates, web feeds such as Rich Site Summary (RSS) feeds, and/or real-time updates from one or more third party information sources.

Additionally, communications subsystem 1224 may also be configured to receive data in the form of continuous data streams, which may include event streams 1228 of real-time events and/or event updates 1230, that may be continuous or unbounded in nature with no explicit end. Examples of applications that generate continuous data may include, for example, sensor data applications, financial tickers, network performance measuring tools (e.g. network monitoring and traffic management applications), clickstream analysis tools, automobile traffic monitoring, and the like.

Communications subsystem 1224 may also be configured to output the structured and/or unstructured data feeds 1226, event streams 1228, event updates 1230, and the like to one or more databases that may be in communication with one or more streaming data source computers coupled to computer system 1200.

Due to the ever-changing nature of computers and networks, the description of computer system 1200 depicted in the figure is intended only as a specific example. Many other configurations having more or fewer components than the system depicted in the figure are possible. For example, customized hardware might also be used and/or particular elements might be implemented in hardware, firmware, software (including applets), or a combination. Further, connection to other computing devices, such as network input/output devices, may be employed. Based on the disclosure and teachings provided herein, other ways and/or methods to implement the various embodiments should be apparent.

As used herein, the terms “about” or “approximately” or “substantially” may be interpreted as being within a range that would be expected by one having ordinary skill in the art in light of the specification.

In the foregoing description, for the purposes of explanation, numerous specific details were set forth in order to provide a thorough understanding of various embodiments. It will be apparent, however, that some embodiments may be practiced without some of these specific details. In other instances, well-known structures and devices are shown in block diagram form.

The foregoing description provides exemplary embodiments only, and is not intended to limit the scope, applicability, or configuration of the disclosure. Rather, the foregoing description of various embodiments will provide an enabling disclosure for implementing at least one embodiment. It should be understood that various changes may be made in the function and arrangement of elements without departing from the spirit and scope of some embodiments as set forth in the appended claims.

Specific details are given in the foregoing description to provide a thorough understanding of the embodiments. However, it will be understood that the embodiments may be practiced without these specific details. For example, circuits, systems, networks, processes, and other components may have been shown as components in block diagram form in order not to obscure the embodiments in unnecessary detail. In other instances, well-known circuits, processes, algorithms, structures, and techniques may have been shown without unnecessary detail in order to avoid obscuring the embodiments.

Also, it is noted that individual embodiments may have been described as a process which is depicted as a flowchart, a flow diagram, a data flow diagram, a structure diagram, or a block diagram. Although a flowchart may have described the operations as a sequential process, many of the operations can be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination can correspond to a return of the function to the calling function or the main function.

The term “computer-readable medium” includes, but is not limited to portable or fixed storage devices, optical storage devices, wireless channels and various other mediums capable of storing, containing, or carrying instruction(s) and/or data. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc., may be passed, forwarded, or transmitted via any suitable means including memory sharing, message passing, token passing, network transmission, etc.

Furthermore, embodiments may be implemented by hardware, software, firmware, middleware, microcode, hardware description languages, or any combination thereof. When implemented in software, firmware, middleware or microcode, the program code or code segments to perform the necessary tasks may be stored in a machine readable medium. A processor(s) may perform the necessary tasks.

In the foregoing specification, features are described with reference to specific embodiments thereof, but it should be recognized that not all embodiments are limited thereto. Various features and aspects of some embodiments may be used individually or jointly. Further, embodiments can be utilized in any number of environments and applications beyond those described herein without departing from the broader spirit and scope of the specification. The specification and drawings are, accordingly, to be regarded as illustrative rather than restrictive.

Additionally, for the purposes of illustration, methods were described in a particular order. It should be appreciated that in alternate embodiments, the methods may be performed in a different order than that described. It should also be appreciated that the methods described above may be performed by hardware components or may be embodied in sequences of machine-executable instructions, which may be used to cause a machine, such as a general-purpose or special-purpose processor or logic circuits programmed with the instructions to perform the methods. These machine-executable instructions may be stored on one or more machine readable mediums, such as CD-ROMs or other type of optical disks, floppy diskettes, ROMs, RAMs, EPROMs, EEPROMs, magnetic or optical cards, flash memory, or other types of machine-readable mediums suitable for storing electronic instructions. Alternatively, the methods may be performed by a combination of hardware and software.

What is claimed is:

1. An imaging system for capturing spatial images of biological tissue samples, the imaging system comprising:
an imaging chamber configured to hold a biological tissue sample placed in the imaging system;
a light source configured to illuminate the biological tissue sample to activate one or more fluorophores in the biological tissue sample;
a Time Delay and Integration (TDI) imager comprising a plurality of partitions, wherein the plurality of partitions are configured to capture images at a plurality of different depths in the biological tissue sample simultaneously during a scan by the TDI imager; and
a controller configured to cause the TDI imager to scan the biological tissue sample.

2. The imaging system of claim 1, wherein the TDI imager is tilted at an angle relative to the biological tissue sample such that focal planes for the plurality of partitions correspond to the plurality of different depths in the biological tissue sample.

3. The imaging system of claim 1, wherein the plurality of partitions on the TDI imager are physically separated by spaces between the plurality of partitions.

4. The imaging system of claim 1, wherein the plurality of partitions on the TDI imager are separated by a row of pixels that are covered.

5. The imaging system of claim 1, wherein the plurality of different depths in the biological tissue sample comprise a plurality of different depth ranges in the biological tissue sample.

6. The imaging system of claim 5, wherein the a partition in the plurality of partitions on the TDI imager corresponds to a depth range in the plurality of different depth ranges, the partition comprises a plurality of pixel rows, and the each of the plurality of pixel rows corresponds to a different depth in the depth range.

7. The imaging system of claim 6, wherein data received from the plurality of pixel rows are combined in a focus-drilling combination to produce an image for the depth range.

8. A method of capturing spatial images of a biological tissue sample, the method comprising:
mounting a biological tissue sample in an imaging chamber of an imaging system;
directing light from a light source to illuminate an area on the biological tissue sample to activate one or more fluorophores in the biological tissue sample; and
scanning the biological tissue sample with a Time Delay and Integration (TDI) imager comprising a plurality of partitions, wherein the plurality of partitions are configured to capture images at a plurality of different depths in the biological tissue sample simultaneously during the scan.

9. The method of claim 8, wherein the plurality of different depths in the biological tissue sample comprise a plurality of different depth ranges in the biological tissue sample, a partition in the plurality of partitions on the TDI imager corresponds to a depth range in the plurality of different depth ranges, the partition comprises a plurality of pixel rows, and each of the plurality of pixel rows corresponds to a different depth in the depth range.

10. The method of claim 9, further comprising combining data received from the plurality of pixel rows in a focus-drilling combination to produce an image for the depth range.

11. The method of claim 9, wherein the depth range is between about 250 nm and about 750 nm.

12. The method of claim 8, wherein the biological tissue sample is between about 2 μm and about 10 μm thick.

13. The method of claim 8, further comprising generating images of the biological tissue sample from each of the plurality of partitions.

14. An imaging system comprising:
a Time Delay and Integration (TDI) imager comprising a plurality of partitions, wherein the plurality of partitions are configured to capture images at a plurality of different depths in a volume simultaneously during a scan by the TDI imager.

15. The imaging system of claim 14, wherein the TDI imager is tilted at an angle relative to the volume such that focal planes for the plurality of partitions correspond to the plurality of different depths in the volume, and the angle is adjustable to fine-tune the plurality of different depths in the volume.

16. The imaging system of claim 14, further comprising a glass cover on the TDI, wherein the glass cover comprises a plurality of sections corresponding to the plurality of partitions, wherein thicknesses of the plurality of sections of the glass cover cause the focal planes for the plurality of partitions be at the plurality of different depths in the volume.

17. The imaging system of claim 14, further comprising a lens in front of the TDI, wherein the lens comprises a plurality of sections corresponding to the plurality of partitions, wherein thicknesses of the plurality of sections of the lens cause the focal planes for the plurality of partitions be at the plurality of different depths in the volume.

18. The imaging system of claim 14, wherein the plurality of partitions of the TDI imager have different heights relative to each other, in the different heights cause the focal planes for the plurality of partitions be at the plurality of different depths in the volume.

19. The imaging system of claim 14, wherein the volume comprises a biological tissue sample.

20. The imaging system of claim 14, further comprising a lens in front of the TDI, wherein the lens comprises a wedge shape, wherein the wedge shape causes the focal planes for the plurality of partitions be at the plurality of different depths in the volume.
